(19) United States
(12) Patent Application Publication
(10) Pub. No.: US 2013/0171198 A1
(43) Pub. Date: Jul. 4, 2013

(54) ANTIMICROBIAL AND VULNERARY ACTION PHARMACEUTICAL COMPOSITION FOR EXTERNAL APPLICATION AND ITS PRODUCTION PROCESS

(71) Applicants: Victor Lvovich Limonov, Moscow (RU); Konstantin Valentinovich Gadul, Novosibirsk (RU); Aleksandr Valerevich Dushkin, Novosibirsk (RU)

(72) Inventors: Victor Lvovich Limonov, Moscow (RU); Konstantin Valentinovich Gadul, Novosibirsk (RU); Aleksandr Valerevich Dushkin, Novosibirsk (RU)

(21) Appl. No.: 13/389,652

(22) PCT Filed: May 11, 2011
(86) PCT No.: PCT/RU11/00322

§ 371 Date: Feb. 9, 2012

(30) Foreign Application Priority Data
Sep. 20, 2010 (EA) 201001506

(51) Int. Cl. A61K 9/14; A61K 31/665

(52) U.S. Cl. CPC A61K 9/143; A61K 31/665
USPC 424/400; 514/99

(57) ABSTRACT

There is proposed herein a process for production of composite antimicrobial and vulnerary preparations for external administration, featuring a higher therapeutic efficiency in case of skin and soft tissues infections treatment. The proposed compositions include an active agent being fosfomycin and finely dispersed nanostructured silica dioxide, with a weight ratio of (25-75 mass. %):(75-25 mass. %) respectively. The mentioned production process includes mixing fosfomycin with finely dispersed nanostructured silica dioxide. The process is distinct in that the mixture of aforementioned substances with the mentioned weight ratio is exposed to mechanical processing by blow impact and abrasive actions until a portion of the fine powder fraction with particles smaller than 5 micrometers, contained in the mixture, increases to at least 40%.

### CROSS-REFERENCE TO RELATED APPLICATIONS

This application is a U.S. national stage application of a PCT application PCT/RU2011/000322 filed on 11 May 2011, whose disclosure is incorporated herein in its entirety by reference, which PCT application claims priority of a EAPO application EA201001506 filed on 20 Sep. 2010.

### FIELD OF THE INVENTION

This invention belongs to antimicrobial pharmaceutical preparations and its' production technologies. It can be used in medicine and veterinary science as a preventive measure and medical maintenance of skin and soft tissues would fever infections, to provide an accelerated wound healing during postoperative period as well as being used in pharmaceutical industry for medicinal products manufacturing.

### BACKGROUND OF THE INVENTION

It is known, that the antibiotic with the international nonproprietary name—fosfomycin, which possesses a wide range antimicrobial action and provides a bacillicidal effect on many gram-positive and gram-negative microorganisms, can be used successfully for skin, soft tissues, bones and joints infections treatment by its' parenteral forms intravenous injections, which is basically fosfomycin sodium salt [1, 2, 3, 4, 5].

It has been discovered that fosfomycin can penetrate into phagocytes (neurophiles and macrophages), stimulate their phagocytic activity and has a bactericidal action on endocellularly located microorganisms [6,7]. It has also been proved that fosfomycin can decrease the inflammatory response terebrant phase as well as penetrate into biofilms generated by multilayered microbial associations, making them vulnerable and loose for other antibiotics [8, 9, 10].

Besides of the qualities mentioned above fosfomycin (in its' parenteral form) in case of local administration is capable of stimulating hemostasis and angiogenesis process, activating monocytes and fibroblasts chemotaxis into inflammation areas, increasing macrophages quantity producing tissular fibronectin; that is why there is a good reason for having patented this antibiotic as a vulnerary external administration pharmacon in powder, wash, crème or any other form [11]. The mentioned patent is the proximate analogue for the proposed pharmaceutical composition and has been accepted as a prototype of this invention.

One of the prototypes' problems is the absence adsorptive and osmolar properties, which prevent the necessary evacuation of the early content and sorption of hystolysis and microbial degradation products, therefore lowering the therapeutic efficiency level.

### DESCRIPTION OF THE INVENTION

The mentioned invention resolves the issue of creating an antimicrobial and vulnerary action pharmaceutical composition for external application on basis of using parenteral form of fosfomycin and finely dispersed nanostructured silica dioxide (BHSiO₂) which possess an increases therapeutic efficiency in case of contagious and inflammatory diseases treatment.

The inventive solution is the use of an antimicrobial and vulnerary action pharmaceutical composition for external application, which contains fosfomycin antibiotic (in powder form) as therapeutic substance as well as finely dispersed nanostructured silica dioxide with a weight ratio—fosfomycin: finely dispersed nanostructured silica dioxide w/w (25-75 mass. %):(75-25 mass. %).

It is also suggested using an antimicrobial and vulnerary action pharmaceutical composition for external application, which contains fosfomycin antibiotic (in powder form) as therapeutic substance as well as finely dispersed nanostructured silica dioxide with a weight ratio—fosfomycin:finely dispersed nanostructured silica dioxide w/w (25-75 mass. %):(75-25 mass. %) and the received mixture is subjected to impact and abrasive actions.

Therapeutic efficiency of the proposed pharmaceutical composition will increase, if it the received mixture is being mechanized by impact and abrasive actions to make the portion of the finely dispersed nanostructured silica dioxide with the dimension ≦5 micron not less than 40%.

To prepare the mentioned pharmaceutical composition, we used fosfomycin (parenteral administration form) produced by a Spanish company “Ercros”. As BHSiO₂ was used “Polysorb” drug (pharmacological group: enterosorbing solution; active substance: colloidal silica dioxide), produced by Russian company CJSC “Polysorb”, containing round shaped silica dioxide nanoparticles (dimension 5-20 nm) combined into aggregates (irregular microparticles) with dimension ≦90 micron (registration number No 001140/01-100908).

The composition formulation choice was based on convertible fosfomycin molecules by micro BHSiO₂ particles sorption process, together with BHSiO₂ particles reduction during its' mixtures mechanical activation with fosfomycin substances by impact abrasive mechanization process.

BHSiO₂ has been chosen because being different from other substances by innocuousness, having absorbing, osmolar and moisture absorbing properties, it is used in medicine for infected wounds treatment and it is also included into the list of well-known vulnerary compositions, which contain fosfomycin antibiotic and are being prepared by a different method comparing to the proposed composition [12, 13,14,15].

Besides, BHSiO₂ has been chosen because SiO₂ nanoparticles being different because of their pharmacologically advantageous biocompatibility, biodistribution, biodegradation and innocuousness properties (not depending from looseness of structure manifestation rate) can serve as antibiotic carriers for endocellular antibiotics delivery into macrophages, which are concentrated in the inflammation areas, i.e. can considerably increase antibiotics concentration in the infected tissues, as well as stimulate the antimicrobial activity of those immune system cells what will lead to an authentic antimicrobial agents therapeutic efficiency increase in case of skin and soft tissues contagious and inflammatory diseases treatment[16, 17].

The stated production process of the previously mentioned pharmaceutical composition by fosfomycin antibiotic powder mixture and BHSiO2 mechanical activation with intensive impact abrasive operations allow to increase the finely dispersed BHSiO2 particles (less than 5 micron) on which fosfomycin molecules are adsorbed and which are mostly phagocyted by macrophages [18].

To achieve this goal, the mixture of the stated above materials in the following weight proportion, fosfomycin antibiotic:BHSiO2 w/w (25-75 mass. %):(75-25 mass. %), is exposed to an intensive impact—abrasive mechanical activation process until the finely divided fraction weight is increased up to 40% of the total weight of the mixture.

The so obtained powder-like composition, containing the finely dispersed BHSiO₂ with convertibly occluded fosfomycin molecules on their surface, can be used as a powder-like composition for external application or as a 1-10% water suspension.

Choosing the ratios of fosfomicin:BHSiO2 equal to (25-75 mass. %):(75-25 mass. %) consequently is determined by the combination of the two following factors:

1) in case of increasing the BHSiO2 content to more than 75% of the composition's weight, its therapeutic efficiency is decreased, because of a decrease of the mass fraction of fosfomicin active pharmaceutical substance;
2) in case of decreasing the BHSi02 content to less than 25% of the composition's weight, its therapeutic efficiency in fact doesn't differ from the basic efficiency of the initial fosfomicin antibiotic.

For obtaining the composition, a mechano-chemical method was used, which contemplates a processing of the solid components mixture by intensive mechanical impacts in the form of pressure and shearing deformations, mostly carried out in different kind of mills, which perform impact (blow) abrasive actions upon the substances. The mixture of the solid fosfomycin antibiotic substance and finely dispersed nanostructured silica dioxide taken in the ratio of (25-75 mass. %):(75-25 mass. %) by weight, are exposed to mechanical activation in grinding ball-mills. The used mixture preparation method helps in a certain way to avoid chemical degradation and achieve powder components full homogeneity in comparison with making the mixture by a simple components mixing, or evaporating their solutions, and as consequence causes a high pharmaceutical activity of the composition.

As a quantitative criterion of the minimum necessary mechanical impact dose it is convenient to use the granulometry method of the composition suspension. It is necessary that the mass fraction of the particles sized less than 5 micrometers be more than 40%. The mechanical processing of powder mixtures is performed in rotary, vibrational, and planetary mills. Suitable balls, rods, and the like can be deployed therein.

Laboratory animals (guiney pigs and rabbits) pharmacological tests of the compositions showed, that the mentioned compositions prepared by the mentioned method have a higher antimicrobial and vulnerary efficiency comparing to the initial fosfomycin.

In such manner, using the mentioned pharmacological compositions and their production process provide the stated below advantages:
1) Clinically significant increase of the effectiveness and quality of the antimicrobial therapy of skin and soft tissues wound fewer, as well as surgical wounds cicatrization period;
2) Ecological safety, lack of wastes and low price of pharmacological production technology.

The proposed invention is illustrated by examples listed below.

### Example No 1

### Powder Like Composition Production: Fosfomycin/BHSiO₂

The mixture of fosfomycin and BHSiO₂ in weight ratio 3:1, 1:1 and 1:3 are being processed in an orbicular rotary mill for 2 and 4 hours. The data of the water suspension granulometric composition (we used a laser Micro-Sizer 201 granulometer) as well as HPLC analysis of the antibiotic content (in % from the initial substance) are listed in the table No 1.

As can be seen from Table No. 1, the chosen conditions of the composition production afford to increase until a certain value (not less than 40% from the total weight) the part of the finely dispersed BHSiO₂ fraction (particles size less than 5 micron) and to avoid the antibiotic chemical degradation.

Fosfomycin sorption rate by BHSiO₂ particles was 20-25%.

TABLE No 1
Water suspensions granulometric composition and fosfomycin
content in different composition variations

| | Dimension and content % | | |
|---|---|---|---|
| | of BHSiO₂ particles* | | Fosfomycin |
| | % < 2 | % < 5 | content |
| Composition content | micron | micron | (%) |
| Initial BHSiO₂ | 0.37 | 5.5 | — |
| Fosfomycin:BHSiO₂ (3:1), | 8.3 | 40.2 | 98 |
| m/a 2 hours | | | |
| Fosfomycin:BHSiO₂ (1:1), | 12.4 | 45.6 | 99 |
| m/a 2 hours | | | |
| Fosfomycin:BHSiO₂ (1:3), | 14.1 | 44.7 | 97 |
| m/a 4 hours | | | |

*finely dispersed nanostructured silica dioxide

### Example No 2

### Determination of the Therapeutic Efficiency of Fosfomycin and Pharmaceutical Compositions

There has been a research of fosfomycin mechanized for 2 hours and composed of a mixture antibiotic/BHSiO₂ in weight ratio 1:1, i.e. (50 mass. %):(50 mass. %).

The experiments were conducted using adult rabbits “Chinchilla” (males, weight 3-3.5 kg) and random bred guiney pigs (males, weight 0.8-0.9 kg) according to the “Regulations for test animals use” (USSR Ministry of health order supplement #755 from 12.08. 1977).

Experimental Models

1. Incision

There have been made several incisions with a sterile scalpel on the left and right side of the depilated coupling area (1.5% novocain solution was used for local anesthesia); incision 2 cm long and 0.8 cm deep with muscular layer seizure for guiney pigs and 2 cm long and 1 cm deep with muscular layer seizure for rabbits.

Each day in 8 days period (starting with the very first day) the wounds of the animals control group was bathed with a sterile normal saline solution, were covered with a sterile tissue fixed with a plaster. The wounds of the test group animals were bathed with a sterile saline solution, then dried and then dusted with fosfomycin sterile powder uniform layer (2-3 mm) or a same layer pharmaceutical composition (for guiney pigs), the wounds of the test rabbits were irrigated with a sterile 2.5% fosfomycin solution or sterile 5% pharmaceutical composition suspension, after that they were dried and covered with a sterile tissue fixed with a plaster.

The regenerative process dynamics was monitored during 9 days. The medics measured the length of the wound open area and made a visual evaluation of wound edges and walls, the presence and character of the exudates, necrosis presence. The test results can be seen in Tables No 2 and No 3.

2. Infected Thermal Burn

There have been made several skin integument burnings with a warmed up on the burner flame metallic applicator on the left and right side of the depilated coupling area (1.5% novocain solution was used for local anesthesia) by attaching it to the skin and holding for 40 seconds. In the center of the ambustial area there has been intracutaneously inserted Staphylococcus aureus (ATCC No 25923 F-49) daily suspension with a 0.1 ml volume in a 10¹⁰ CFU/ml dosage.

Each day in 13 days period (starting with the very first day) the burnings of the animals control group was bathed with a sterile normal saline solution, dried and covered with a sterile tissue fixed with a plaster. The burnings of the test animals were irrigated with a sterile 2.5% fosfomycin solution or sterile 5% pharmaceutical composition suspension, after that they were dried and covered with a sterile tissue fixed with a plaster.

The burnings condition dynamics was monitored during 14 days. The medics measured the burning wound surface and necrosis area in the center of the burning wound. You may see the results in the table No 4.

The statistical data processing has been made with a program Statistica 6.0. The experimental data are presented as median (Me), Low and High quartile (LQ-HQ), the difference authenticity has been calculated using Student t-criterion with p<0.05 values.

Results

1. In case of incision experiments after 48 hours the guiney pigs had results mentioned below: even wound edges, clear bottom, visible bands and lateral oblique muscles muscular layer, in the wound center there was a sanioserous exudates minute amount, no microbial contamination has been noted. From day 2 to day 9 there has been noted an open wound area reduction. This parameter has been used as a regeneration exponent.

From the data shown in Table No 2 it can be seen that fosfomycin powder forms and pharmaceutical compositions fosfomycin:BHSiO₂ (w/w 1:1) authentically increase incisions regeneration process comparing to the control group, taking into consideration the fact that from day 6 the composition therapeutic efficiency was authentically higher than fosfomycin.

TABLE No 2
Fosfomycin and pharmaceutical composition (powder form)
influence on incisions regeneration process for
guiney pigs in case of external application.

| | | Incision length (cm) | | |
|---|---|---|---|---|
| Test groups | | Me (LQ-HQ)* | | |
| (n—animals q-ty) | | Day 2 | Day 6 | Day 9 |
| 1 | Control | 1.3 | 1.1 | 0.6 |
| | (n = 8) | (1.1-1.8) | (0.9-1.3) | (0.4-0.7) |
| 2 | Fosfomycin | 0.9 | 0.5 | 0.3 |
| | (n = 10) | (0.7-1.3) | (0.3-0.6) | (0.2-0.4) |
| | | | P₁₋₂ < 0.02 | P₁₋₂ < 0.05 |
| 3 | Fosfomycin:BHSiO₂ | 0.6 | 0.3 | 0.15 |
| | (1:1), m/a for 2 hours | (0.5-0.7) | (0.2-0.4) | (0.1-0.2) |
| | (n = 10) | P₁₋₃ < 0.01 | P₁₋₃ < 0.01 | P₁₋₃ < 0.01 |
| | | | P₂₋₃ < 0.05 | P₂₋₃ < 0.02 |

*median, low and high quartiles

2. In case of incision experiments after 48 hours the rabbits had results mentioned below: even wound edges, clear bottom, visible bands and lateral oblique muscles muscular layer, in the wound center there was a sanioserous exudates minute amount, no microbial contamination has been noted. In the next few days the wounds had no differences according to the mentioned parameters (for all test groups). From day 2 to day 9 there has been noted an open wound area reduction. This parameter has been used as a regeneration exponent.

From the data shown in Table No 3 it can be seen that 2.5% fosfomycin solution and 5% pharmaceutical composition water suspension fosfomycin:BHSiO₂ (w/w 1:1) authentically increase incisions regeneration process comparing to the control group, taking into consideration the fact that from day 6 the composition therapeutic efficiency was authentically higher than fosfomycin.

TABLE No 3
Fosfomycin and pharmaceutical composition (2.5% solution and 5% suspension
respectively) influence on incisions regeneration process for rabbits in case of external
application.

| | | Incision length (cm) | | |
|---|---|---|---|---|
| Test groups | | Me (LQ-HQ)* | | |
| (n—animals q-ty) | | Day 2 | Day 6 | Day 9 |
| 1 | Control | 0.6 | 0.5 | 0.35 |
| | (n = 8) | (0.5-0.7) | (0.4-0.6) | (0.3-0.4) |
| 2 | Fosfomycin | 0.5 | 0.4 | 0.25 |
| | (n = 8) | (0.4-0.6) | (0.3-0.5) | (0.2-0.3) |
| | | | | P₁₋₂ < 0.05 |
| 3 | Fosfomycin:BHSiO₂ (1:1), | 0.45 | 0.25 | 0.1 |
| | m/a for 2 hours | (0.4-0.5) | (0.2-0.3) | (0.05-0.15) |
| | (n = 8) | | P₁₋₃ < 0.01 | P₁₋₃ < 0.01 |
| | | | P₂₋₃ < 0.05 | P₂₋₃ < 0.01 |

*median, low and high quartiles

3. In case of infected thermal burn wound after 24 hours after the experiment has been started the guiney pigs had a notable skin integument induration in the thermal burn area, there has been an edema, there has been noted an opened and dried wheal as well as sanioserous secretion. The edges of the burning wound are clearly limited from the surrounding unaffected skin. Starting from the experiment day 3 there has been noted a necrosis area in the center of the burning. Starting with experiment day 4 there has been noted a decrease of the burnt area and necrosis area augmentation (with its' further decrease).

From the data shown in Table No 4 it can be seen that 2.5% fosfomycin solution and 5% pharmaceutical composition water suspension fosfomycin:BHSiO₂ (w/w 1:1) authentically increase infected S. aureus thermal burn regeneration process comparing to the control group, taking into consideration the fact that from day 6 the composition therapeutic efficiency was authentically higher than in case of using fosfomycin.

TABLE No 4
Fosfomycin and pharmaceutical composition (2.5% solution and 5% suspension
respectively) influence on infected S. aureus thermal burn regeneration process for guiney
pigs in case of external application.

| Test groups | | Thermal burn area/necrosis area (cm²) | | | |
|---|---|---|---|---|---|
| (n—animals q-ty) | | Day 2 | Day 6 | Day 9 | Day 14 |
| 1 | Control | 1.2/0.0 | 1.1/0.5 | 1.1/0.5 | 1.0/0.4 |
| | (n = 8) | | | | |
| 2 | Fosfomycin | 1.2/0.0 | 0.8/0.3 | 0.7/0.25 | 0.4/0.2 |
| | (n = 10) | | P₁₋₂ < 0.05/P₁₋₂ < 0.05 | P₁₋₂ < 0.05/P₁₋₂ < 0.02 | P₁₋₂ < 0.01/P₁₋₂ < 0.01 |
| 3 | Fosfomycin: | 1.2/0.0 | 0.5/0.15 | 0.4/0.1 | 0.15/0.05 |
| | BHSiO₂ | | P₁₋₃ < 0.01/P₁₋₃ < 0.01 | P₁₋₃ < 0.01/P₁₋₃ < 0.01 | P₁₋₃ < 0.01/P₁₋₃ < 0.01 |
| | (1:1), m/a for | | P₂₋₃ < 0.05/P₂₋₃ < 0.01 | P₂₋₃ < 0.05/P₂₋₃ < 0.01 | P₂₋₃ < 0.01/P₂₋₃ < 0.01 |
| | 2 hours | | | | |
| | (n = 10) | | | | |

Therefore, basing on the received test results (using incision and infected S. aureus thermal burn models), we can come to the conclusion that the suggested pharmaceutical composition of antimicrobial and vulnerary action for external application (fosfomycin/BHSiO₂) has a considerably increased therapeutic effect in case of soft tissues and skin infections treatment comparing to the initial fosfomycin (prototype of the mentioned invention).

### USED LITERATURE

1. Frossard M., Joukhadar C., Erovic B. M. et al. Distribution and antimicrobial activity of fosfomycin in the interstitial fluid of human soft tissues//Antimicrob. Agents Chemother.-2000.-Vol. 44.-P. 2728-2732.
2. Legat F. J., Maier A., Dittrich P. et al. Penetration of fosfomycin into inflammatory lesions in patients with cellulitis or diabetic foot syndrome//Antimicrob. Agents Chemother.-2003.-Vol. 47.-P.371-374.
3. Sauermann R., Karch R., Langenberger H. et al. Antibiotic abscess penetration: fosfomycin levels measured in pus and simulated concentration-time profiles//Antimicrob. Agents Chemother.-2005.-Vol. 49.-P. 4448-4454.
4. Schintler M. V., Traunmuller F., Metzler J. et al. High fosfomycin concentrations in bone and peripheral soft tissue in diabetic patients presenting with bacterial foot infection//J. Antimicrob. Chemother.-2009.-Vol. 64.-P.574-578.
5. Fernandez-Valencia J. E., Saban T., Canedo T., Olay T. Fosfomycin in osteomyelitis//Chemotherapy.-1976.-Vol. 22.-P.121-134.
6. Traub W. H. Interactions of antimicrobial drugs and combined phagocytic/serum bactericidal activity of defibrinated human blood against Serratia marcescens.III. Beta-lactam antibiotics and fosfomycin//Chemotherapy.-1983.-Vol. 29.-P.48-57.
7. Perez-Fernandez P., Herrera I., Martinez P. et al. Enhancement of the susceptibility of Staphylococcus aureus to phagocytosis after treatment with fosfomycin compared with other antimicrobial agents//Chemotherapy.-1995.-Vol. 41.-P.45-49.
8. Matsumoto T., Tateda K., Miyazaki S. et al. Fosfomycin alters lipopolysaccharide-induced inflammatory cytokine production in mice//Antimicrob. Agents Chemother.-1999.-Vol. 43.-P. 697-698.
9. Cai Y., Fan Y., Wang R. et al. Synergistic effects of aminoglicosides and fosfomycin on Pseudomonas aeruginosa in vitro and biofilm infections in a rat model//J. Antimicrob. Chemother.-2009.-Vol. 64.-P. 563-566.
10. Zeitlinger M., Marsik C., Steiner I. et al. Immunomodulatory effects of fosfomycin in an endotoxin model in human blood//J. Antimicrob. Chemother.-2006.-Vol. 59.-P. 219-223.
11. European Patent EPO470431.
12. Clinical chemistry and silica dioxide clinical use//Under the editorship of A. A. Chuiko, academic of NAS Ukraine-Kiev:<<Naukova dumka>>, 2003.-416 p.
13. Patent No. 32088 UA (Ukraine).
14. Patent No. 33629 UA (Ukraine).
15. Chuiko A., Pentyuk A., Shtat'ko E., Chuiko N. Medical aspects of application of highly disperse amorphous silica//Surface Chemistry in Biomedical and Environmental Science. Edited by J. P. Blitz and V. Gun'ko.II. Mathematics, Physics and Chemistry-2006.-Vol. 228.-P.191-204.
16. Seleem M. N., Munusamy P., Ranjan A et al. Silica-antibiotic hybrid nanoparticles for targeting intracellular pathogens//Antimicrob. Agents Chemother.-2009.-Vol. 53.-P.4270-4274.
17. Waters K. M., Masiello L. M., Zangar R. C. et al. Macrophage responses to silica nanoparticles are highly conserved across particle sizes//Toxicological Sciences.-2009.-Vol. 107.-P. 553-569.
18. Hamilton R. F., Thakur S. A., Mayfair J. K., Holian A. MARCO mediates silica uptake and toxicity in alveolar macrophages from C57BL/6 mice//J. Biological Chemistry.-2006.-Vol. 281.-P. 34218-34226.

1. A pharmaceutical composition for antimicrobial and vulnerary use for external application, wherein said composition is made in the form of powder; said composition including: a first amount of fosfomycin antibiotic acting as a therapeutic agent, and a second amount of finely dispersed nanostructured silica dioxide; wherein said composition is characterized by a weight ratio of the first amount to the second amount, and said weight ratio constituting: (25-75 mass. %):(75-25 mass. %).

2. The composition according to claim 1, wherein said finely dispersed nanostructured silica dioxide is composed of particles; said finely dispersed nanostructured silica dioxide includes a portion of at least 40% thereof, wherein said portion substantially consists of said particles having a size not exceeding 5 micrometers.

3. A process of production of an antimicrobial and vulnerary pharmaceutical composition for external administration comprising the steps of:
providing a first amount of fosfomycin in the form of powder;
providing a second amount of finely dispersed nanostructured silica dioxide in the form of powder;
mixing said first amount with said second amount in a weight ratio of (25-75 mass. %):(75-25 mass. %), thereby obtaining a mixture; and
subjecting said mixture to mechanical processing by means of blow-abrasive actions.

4. The process according to claim 3, wherein said finely dispersed nanostructured silica dioxide is composed of particles; said blow-abrasive actions are resulted in that said finely dispersed nanostructured silica dioxide includes a portion of at least 40% thereof, wherein said portion substantially consists of said particles having a size not exceeding 5 micrometers.
